(19) United States
(12) Patent Application Publication
(10) Pub. No.: US 2023/0285768 A1
(43) Pub. Date: Sep. 14, 2023

(54) COMPACT BATTERY-POWERED REPETITIVE TRANSCRANIAL MAGNETIC STIMULATION

(71) Applicants: Kevin MURPHY, Oakland, CA (US); David GOUGH, Oakland, CA (US); The Regents of the University of California, Oakland, CA (US)

(72) Inventors: Kevin Murphy, San Diego, CA (US); Vincent Leung, McGregor, TX (US); Peter Asbeck, Del Mar, CA (US); Mark Stambaugh, La Jolla, CA (US); Milan Makale, San Diego, CA (US); David Gough, Solana Beach, CA (US); Shaghayegh Abbasi, Portland, OR (US)

(21) Appl. No.: 18/005,811

(22) PCT Filed: Jul. 16, 2021
(86) PCT No.: PCT/US2021/042057

§ 371 Date: Jan. 17, 2023

Related U.S. Application Data
(60) Provisional application No. 63/052,885, filed on Jul. 16, 2020.

(51) Int. Cl. A61N 2/02; A61N 2/00

(52) U.S. Cl. CPC A61N 2/02; A61N 2/006

(57) ABSTRACT

A portable therapeutic device and a method. The device includes an energy storage device coupled to a power supply. The energy storage device operates during a predetermined number of charge-discharge cycles. During a charge portion of each charge-discharge cycle, the energy storage device receives and stores energy from the power supply. During a discharge portion of each charge-discharge cycle, the energy storage device discharges stored energy. The device also includes a magnetic field generation device coupled to the energy storage device to repeatedly generate one or more magnetic field pulses during a predetermined period of time during the discharge portion of each charge-discharge cycle of the energy storage device. Each magnetic field pulse has a predetermined magnetic field strength. The generated magnetic field pulses cause generation of an electric field having a predetermined strength, thereby generating a desired therapeutic effect in a subject.

### CROSS-REFERENCE TO RELATED APPLICATIONS

The present application claims priority to U.S. Provisional Patent Appl. No. 63/052,885 to Murphy et al., filed Jul. 16, 2020, and entitled “Compact Battery-Powered Repetitive Transcranial Magnetic Stimulation,” and incorporates its disclosure herein by reference in its entirety.

### TECHNICAL FIELD

The subject matter disclosed herein relates to a repetitive transcranial magnetic stimulation.

### BACKGROUND

Transcranial magnetic stimulation (TMS) is a noninvasive procedure that uses magnetic fields to stimulate nerve cells in the brain to improve symptoms of depression. During a repetitive TMS, an electromagnetic coil is placed on patient's head and repetitive magnetic pulses are delivered to the patient. The pulses cause stimulation of nerve cells of patient's brain in regions that may have a decreased activity. For example, the stimulated region may be involved in controlling mood and/or depression.

### SUMMARY

In some implementations, the current subject matter relates to a portable therapeutic device The device may include an energy storage device (e.g., a capacitor, etc.) coupled to a power supply (e.g., a battery, a power supply). The energy storage device may be configured to store energy received from the power supply. The energy storage device may be further configured to operate during a predetermined number of charge-discharge cycles. During a charge portion of each charge-discharge cycle, the energy storage device may be configured to receive and store energy from the power supply. During a discharge portion of each charge-discharge cycle, the energy storage device may be configured to discharge stored energy. The portable therapeutic device may be configured to accommodate currents of at least 800 Amperes (e.g., 800-2500 A) and a voltage supply of at least 200 Volts (e.g., 200-400 V).

The portable therapeutic device may further include a magnetic field generation device (e.g., inductive coil, etc.) that may be coupled to the energy storage device and configured to repeatedly generate one or more magnetic field pulses in a plurality of magnetic field pulses during a predetermined period of time (e.g., 13-15 minutes). Each magnetic field pulse may have a predetermined magnetic field strength. The pulses may be generated during the discharge portion of each charge-discharge cycle of the energy storage device. Further, the pulses may include single phasic and/or biphasic magnetic pulses occurring as pulse trains over a predetermined frequency (e.g., 10 Hz, etc.) over a predetermined period of time (e.g., 10 seconds, etc.), each pulse train including a predetermined number of pulses (e.g., 100 pulses), where trains may include bursts that may be separated by a predetermined period of time, as discussed above. The therapeutic device may be configured to generate one or more (e.g., 20) pulse trains separated by a predetermined inter-train interval (e.g., 30 seconds, etc.). For example, the therapeutic device may be able to generate up to 4000 pulses or more during any therapeutic/treatment time period (e.g., 13-15 minutes).

The generated magnetic field pulses may be configured to cause generation of an electric field having a predetermined strength, thereby generating a desired therapeutic effect in a subject.

In some implementations, the current subject matter may be configured to include one or more of the following optional features. As stated above, the energy storage device may include a capacitor. The magnetic field generation device may include an inductive coil having a conductive wire, the conductive wire is configured to be wound. The inductive coil may be configured to have a predetermined shape. The predetermined shape may include at least one of the following: a circular shape, a figure-8 shape, an oval shape, an elliptical shape, a butterfly shape, a double butterfly shape, a triple butterfly shape, an H-coil shape, a regular shape, an irregular shape, and any combination thereof.

In some implementations, the inductive coil may include at least one of the following parameters: a predetermined length, a predetermined number of winding turns of the conductive wire, a predetermined radius of one or more winding turns of the conductive wire, a thickness of the conductive wire, and any combination thereof. The predetermined magnetic field strength may be determined using at least one of the inductive coil parameters. The predetermined length may be in a range of approximately 50 mm to 150 mm.

In some implementations, the predetermined strength of the generated electric field may be a range of approximately 50 V/m to 120 V/m. In particular, the predetermined strength of the generated electric field may be approximately 65 V/m.

In some implementations, the power supply may be rechargeable.

In some implementations, the magnetic field generation device may be configured to generated one or more magnetic field pulses as a result of a predetermined current received from the energy storage device. The predetermined current may be in a range of approximately 800 A to 2500 A.

In some implementations, the magnetic field pulses may be generated at a predetermined frequency, where the predetermined frequency may be determined based on the desired therapeutic effect.

In some implementations, the therapeutic device may include a voltage step-up device coupled to the power supply and the energy storage device and configured to increase voltage being supplied by the power supply to the energy storage device. The voltage supplied to the energy storage device may be greater than approximately 200 V.

In some implementations, the therapeutic device may include a printed circuit board for positioning at least one of the power supply, the energy storage device, the magnetic field generation device, and any combination thereof.

In some implementations, the magnetic field pulses may be configured to be applied to the subject from a predetermined distance. The predetermined distance may be in a range of 1.5 cm to 2.5 cm. Further, the therapeutic effect may include a repetitive transcranial magnetic stimulation. The predetermined magnetic field strength may be greater than 100 mT.

Implementations of the current subject matter can include, but are not limited to, systems and methods consistent including one or more features are described as well as articles that comprise a tangibly embodied machine-readable medium operable to cause one or more machines (e.g., computers, etc.) to result in operations described herein. Similarly, computer systems are also described that may include one or more processors and one or more memories coupled to the one or more processors. A memory, which can include a computer-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including but not limited to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein may be apparent from the description and drawings, and from the claims. While certain features of the currently disclosed subject matter are described for illustrative purposes in relation to an enterprise resource software system or other business software solution or architecture, it should be readily understood that such features are not intended to be limiting. The claims that follow this disclosure are intended to define the scope of the protected subject matter.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 is a block diagram of an exemplary rTMS system, according to some implementations of the current subject matter;

FIG. 2 illustrates exemplary implementations of the coil, according to some implementations of the current subject matter;

FIG. 3 illustrates an exemplary rechargeable rTMS system, according to some implementations of the current subject matter;

FIG. 4a illustrates an exemplary switching inductor-capacitor circuit that may be used by the system shown in FIG. 3, according to some implementations of the current subject matter;

FIG. 4b illustrates an exemplary plot showing charging/discharging of a capacitor of the system shown in FIG. 3, according to some implementations of the current subject matter;

FIG. 4c illustrates an exemplary plot showing exemplary experimental values determined using the above experimental system, according to some implementations of the current subject matter;

FIG. 5a illustrates an exemplary voltage step-device, according to some implementations of the current subject matter;

FIG. 5b illustrates an exemplary timing diagram, according to some implementations of the current subject matter;

FIG. 6 illustrates exemplary experimental measured transient waveforms for V_{C}(t), I_{L}(t) and the B-field, generated in accordance with implementations of the current subject matter system;

FIG. 7 illustrates an exemplary system, according to some implementations of the current subject matter; and

FIG. 8 illustrates an exemplary method, according to some implementations of the current subject matter.

### DETAILED DESCRIPTION

One or more implementations of the current subject matter relate to methods, systems, articles of manufacture, and the like that may, among other possible advantages, provide for systems, devices, and/or methods for providing a portable repetitive transcranial magnetic stimulation device, system and associated methods.

Repetitive transcranial magnetic stimulation (rTMS) has rapidly expanded as a safe and effective therapeutic intervention for treatment resistant depression, anxiety, as well as potentially other medical conditions, and beneficially modulating neuronal activity in brain cortical regions. Existing rTMS clinical systems are large, heavy, complex, and costly, so disadvantaged areas and populations, and small medical facilities and environments, may have inadequate rTMS availability. Moreover, many patients cannot visit a standard rTMS clinic more than two or three times weekly, and currently, most patients do not get treated on weekends. Large systems may also not be feasible for specialized applications, such as, for example, circadian rhythm normalization during space travel to Mars. The current subject matter system relates to a miniaturized, portable, and affordable rTMS system that may expand access to a wide array of subjects. Further, current subject matter's portable personal rTMS device may facilitate multiple daily treatments to enhance therapeutic response. Moreover, a compact magnetic inductor coil, also referred to as a head coil, may be integrated with various electroencephalography (EEG) acquisition array and a controller devices to form a closed loop therapeutic system. For example, one or more inductor coils and/or an array of inductor coils (having any number of turns, any thickness of the coil wire, overall size, etc.) may be integrated into a device (e.g., a head cap, a helmet, and/or any other medical device) that may be used for treatment of a patient (e.g., by creating a closed loop rTMS-EEG treatment system). Alternatively, or in addition to, the current subject matter device may be portable, such as, for example, it may be carried on a belt, in a backpack, in a pouch, etc.

In some implementations, the current subject matter may be configured to include a scaled-down rTMS driving circuit and power system based on a head coil design, and may be further based on the following factors: (1) a required magnetic field strength and associated E-field amplitude to be generated by the head coil, and (2) physical properties of the head coil, such as size, number of turns, and inductance. These factors along with therapeutic E-field intensity requirements (e.g., that may be specific for treatment of various medical conditions) may be configured to define topology of the head coil's driving circuitry. In particular, the current subject matter's scaled-down rTMS system may be characterized by a small size and weight, inductance in a range compatible with driving circuits having limited voltage and current handling capability, and comparatively low strength but rapidly changing magnetic fields sufficient to elicit therapeutic levels of stimulation at the human brain cortex. Further, the current subject matter system may be configured to generate/use pulsed magnetic field rise time, intensity, and sustained pulsing, which may be used for validation of design parameters for a miniaturized rTMS head coil. Some examples of the shapes of the head coil may include, but are not limited, to circular shapes, figure-8 shapes, oval shapes, elliptical shapes, butterfly shapes, double/triple butterfly shapes, H-coil shapes, regular shapes, irregular shapes, and/or any other shapes. For example, the figure-8 may offer a comparatively high degree of electric field focality for rTMS. Based on requisite E-field strength and theoretical considerations, the exemplary head coil lengths may be in a range of 50 mm to 150 mm (e.g., 50 mm, 76 mm, 80 mm, 100 mm and 150 mm) or any other desired lengths. Using the head coil, the current subject matter's rTMS system may be driven using predetermined voltage ranges that may be supplied from a power source coupled to the rTMS system. By way of a non-limiting experimental example, when the head coil (e.g., 76×38 mm head coil) is driven at 300V, the head coil may generate an E-field of 65 V/m at 1.5 cm (e.g., 1.5-2.5 cm) depth from the coil bottom surface, which is approximately 65% of the E-field intensity produced by an existing clinical 1.4 T rTMS device operating at 60% of peak power. By way of a non-limiting example, the head coil may be less than 80×50 mm (where the first parameter (i.e., 80 mm) indicates an overall length of the coil wire when unwound, and the second parameter (i.e., 50 mm) indicates a width of the coil when the wire is wound in a particular form (e.g., circular, figure-8, etc.). Further, the current subject matter's device may be configured to induce an electric field of at least approximately 50 V/m and up to 120 V/m at at least a distance of 1.5 cm (or any other desired distance) from the surface of the coil. The current subject matter's device may also be able to accommodate a current supply in a range of approximately 800 A to 2500 A, and a voltage supply of approximately 200 V to 400 V.

Advantageously, the above head coil weighs only 12.6 g (0.4 oz), as compared to 1.8 to 3.9 Kg (4-8.6 lbs.) for existing systems. In some exemplary, non-limiting implementations, an overall weight of the current subject matter's rTMS device (e.g., device implementing a single head coil) may be less than 7 lb (or even lighter), where the head coil's weight may be less than 100 g (or lighter).

Further, the current subject matter device may be configured to include a rechargeable battery that may be capable of generating between approximately 10 volts and 100 volts (and/or any other desired values). Such voltage may be configured to support at least one full therapeutic treatment, such as, for instance, a treatment performed a predetermined period of time (e.g., during at least 13-15 minutes). Alternatively, or in addition to, the current subject matter device's battery may be configured to support any number of treatments. It may also be recharged on as needed basis.

In some exemplary implementations, the current subject matter's device may be configured, on a single charge, to generate one or more sequential single phasic and/or biphasic magnetic pulses having one or more microsecond bursts (e.g., 280 microsecond (μs) or any other value) bursts that may be separated by a predetermined time interval (e.g., 10 msec, etc.). These pulses may be configured to occur at a predetermined frequency (e.g., 10 Hz, etc.) over a certain time period (e.g., 10 seconds, etc.) for trains of containing a predetermined number of pulses (e.g., 100 pulses, etc.). By way of a non-limiting example, the current subject matter's device may be configured to generate 20 trains with an inter-train interval of 30 seconds, thereby generating a total of 2000 pulses. As can be understood any number of magnetic pulses may be supported by the device (e.g., 4000 pulses, etc.).

Additionally, to ensure that the device is compliant with various regulations that may be associated with rTMS device, the current subject matter may be encapsulated in a housing that may be manufactured from thermally-insulating material to prevent heat transfer to the patient. The housing may also include various cooling mechanisms (e.g., a cooling liquid, cooling elements, etc.) to reduce an amount of heat that may be generated during operation of the device.

In that regard, in some implementations, the current subject matter relates to a portable, compact, battery-powered repetitive transcranial magnetic stimulation (rTMS) system. The system may be configured to include a circuitry that may have, among other components, an inductive head coil that may be characterized by one or more of the following parameters: a size of the coil, a type of the coil, a type of wire used in the coil, a thickness of the wire used in the coil, a specific winding of the coil, and/or any other parameters. The system may further include a rechargeable power source (e.g., a battery) for supplying power to a capacitor, which may be configured to discharge through the inductive coil. The inductive coil, in turn, may be configured to generate a magnetic field (e.g., a train of magnetic field pulses) that may be applied to the brain of the patient through one or more switch-type device (e.g., insulated-gate bipolar transistor (IGBT) based switches). The application of magnetic field pulses may be applied to the patient at a predetermined distance. The generated magnetic field pulses may cause stimulation of neurons in the brain of the patient. The pulses may be repetitive and may be applied for a predetermined period of time. In some exemplary, non-limiting implementations, as stated above, the current subject matter system may be configured to generate a 10 Hz magnetic pulse train with a peak flux density of 100 mT at 1.5-2.5 cm distance. The current subject matter system may be a portable, inexpensive, and lightweight rTMS system capable of generating therapeutic levels of current, pulse rise time, and number of pulses. For example, the generated magnetic field may be approximately 0.1 Tesla which is sufficiently close to therapeutic intensity, whereby the current subject matter system's driving circuitry may be scalable to support much stronger fields. The compact, battery-powered rTMS system, as disclosed herein, may have various uses, including, but not limited to, rTMS treatment at home, in a clinic, on a vessel, at a field hospital, on an ambulatory basis, and/or at any desired location or way.

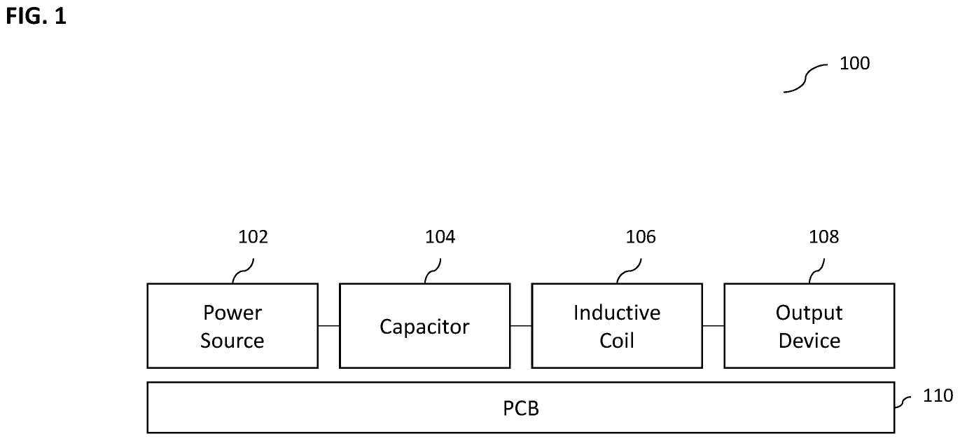

FIG. 1 is a block diagram of an exemplary rTMS system 100, according to some implementations of the current subject matter. The system 100 may include a power source 102, a capacitor 104, an inductive head coil 106, and an optional output device 108. One or more components 102-108 may be integrated on a printed circuit board (or any other substrate) 110. The output device 110 may include one or more sensors, displays, computing components, electrical components, and/or any other types of components.

In some implementations, the rTMS system 100 may be configured to include a rTMS driving circuit that may include an inductor-capacitor (LC) resonator. The capacitor (e.g., capacitor 104) may be initially charged to a high DC voltage through a power supply (e.g., power source 102) and/or a boost DC/DC converter connected to a lower-voltage battery. When the charged capacitor is discharged through the inductor (e.g., inductor head coil 106), the voltage may drive a large current through the coil 106, converting the electrical energy stored on the capacitor 104 into magnetic energy stored on the inductor 106. As the capacitor voltage falls to zero, the inductor current may peak. At that point in time, maximum magnetic field may be built up around the coil 106.

In some implementations, the inductor coil 106 may be implemented as a shaped coil (e.g., circular, figure-8, etc.) with multiple turns. The magnetic field of a single-turn coil is determined by, using Biot-Savart law:

$$B = \frac{{μ}_{o} I}{2 r} \cdot \frac{1}{{( 1 + \frac{{z}^{2}}{{r}^{2}} )}^{3 / 2}} \vert \to \qquad ( 1 )$$

where μ₀ is the permeability (4π10-7 H/m), I is the current (in A), and r and z (both in m) are the coil radius and the vertical distance (where the B-field is measured), respectively. For multiple-turn (N) coil design, the total magnetic field is determined by a linear sum (NB).

In some implementations, the inductor coil may be characterized by at least one of the following parameters: coil size, coil shape, and/or peak current. The following performance metrics may be used to determine the effect of each of these parameters on an electric field generated by the device 100, and may include a maximum electric field (Eₘₐₓ), half-value depth (d_{1/2}), i.e., a radial distance from cortical surface to the deepest point inside the cortex where the E-field value is half of its maximum (Eₘₐₓ), and a half-value tangential spread (S_{1/2}), i.e., where this metric is related to focality of E-field, and it may be defined as follows: S_{1/2}=V_{1/2}/d_{1/2}. Here, V_{1/2} is the half-value volume, defined as the volume of the brain region that is exposed to an electric field stronger than half of the maximum electric field, where the lower S_{1/2}, the more focal the E-field.

In some exemplary experimental implementations, the coil 106 may have a figure-8 shape and may include small compact coils on the order of 4-7 cm in total length (as can be understood any other lengths are possible, if desired). The figure-8 coil may include two circular coils adjacent to each other and may use superposition to generate a larger net electric field in the center. This comparatively high degree of focality may offer distinct advantages in terms of localizing stimulation to discrete areas of the brain. This may also be used to change an orientation of the net magnetic field by altering the phases of the two coil currents. Table 1 illustrates exemplary experimental current pulse results, where figure-8 coil is used, for a monophasic current pulse with a peak value of 1500 A and a pulse width of 70 μsec. The size specified for figure-8 coil it is the largest dimension (e.g., twice the diameter of each circular part). The number of turns in the coil, in this experimental implementation is 9 (as can be understood, any number of turns may be used). The results illustrate how the coil size affects maximum E-field, half-value depth and half-value tangential spread, which are indicators of depth and focality of the E-field.

TABLE 1
Exemplary experimental results for FIG.-8 coil.

| Coil Size L (mm) | Eₘₐₓ(V/m) | d_{1/2}(m) | S_{1/2}(m²) |
|---|---|---|---|
| 36 | 124.09 | 3.30E−03 | 8.21E−5 |
| 50 | 138.15 | 5.00E−03 | 2.34E−04 |
| 100 | 167.44 | 1.37E−02 | 9.44E−04 |
| 150 | 176.06 | 1.16E−02 | 1.25E−03 |

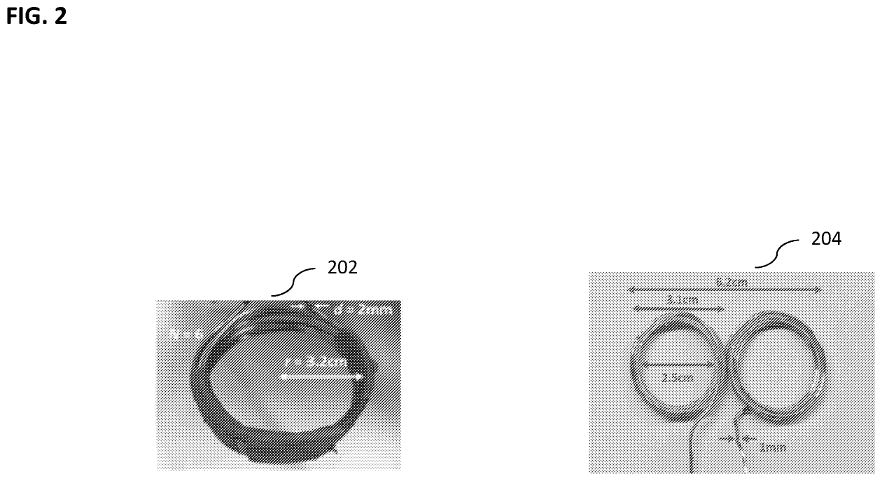

FIG. 2 illustrates exemplary implementations of the coil 106, according to some implementations of the current subject matter. The coil 106 may have a circular shape 202, a figure-8 shape 204, and/or any other desired shape. By way of a non-limiting example, the circular shape 202 may be characterized by the following parameters: number of turns 6, coil radius of 32 mm, and coil wire (e.g., gauge 10) diameter (d) of 2 mm. This circular shape coil may be configured to carry 1.5 kA B-field, an optimized field strength >100 mT whereby the coil may be positioned at 2 cm distance from the patient's head. The coil inductance may be approximately 3.4 μH. In some implementations, the inductance value parameter may be used for determining the shape and/or amplitude of the induced current pulse.

The inductance and resistance of the coil (solenoid) may be determined using the following equations (2)-(3):

$$L \approx \frac{{μ}_{o} {N}^{2} \cdot π {r}^{2}}{N \cdot d + 0.9 r} \qquad ( 2 )$$

$$R = ρ \cdot \frac{2 π r \cdot N}{π {( d / 2 )}^{2}} \qquad ( 3 )$$

where ρ is the resistivity (e.g., 1.72×10-8 Ωm for Cu).

In the figure-8 shaped coil 106 (shown in FIG. 2), Table 2 illustrates an experimental effect of current peak and pulse width parameters, for a monophasic current pulse, for the rTMS system 100. In this case, the coil size was 50 mm diameter of each circular half for the figure-8 shaped coil. As shown in Table 2, the half-value depth (d_{1/2}) and half-value tangential spread (S_{1/2}) may be dependent on the coil size, and changing the electric current characteristics may affect the maximum E-field.

TABLE 2
Coil current pulse peak and width.

| Coil Current | | | | |
|---|---|---|---|---|
| Peak (A) | Pulse Width (μs) | Eₘₐₓ(V/m) | d_{1/2}(m) | S_{1/2}(m²) |
| 1000 | 100 | 46.051 | 5.00E−03 | 1.59E−04 |
| | 50 | 92.103 | 5.00E−03 | 1.59E−04 |
| 1500 | 100 | 69.077 | 5.00E−03 | 1.59E−04 |
| | 50 | 138.15 | 5.00E−03 | 1.59E−04 |
| 2500 | 100 | 115.13 | 5.00E−03 | 1.59E−04 |
| | 50 | 230.26 | 5.00E−03 | 1.59E−04 |
| 5000 | 100 | 230.26 | 5.00E−03 | 1.59E−04 |
| | 50 | 460.51 | 5.00E−03 | 1.59E−04 |

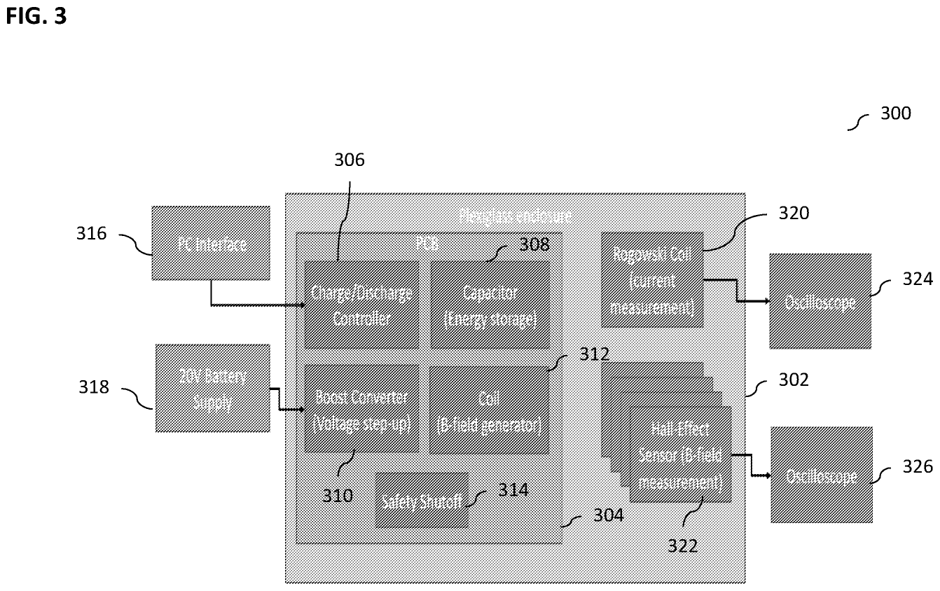

FIG. 3 illustrates an exemplary rechargeable rTMS system 300, according to some implementations of the current subject matter. The system 300 is similar to the system 100 shown in FIG. 1. The system 300 may be configured to include an enclosure 302 that may be configured to include a printed circuit board (PCB) 304, a current measurement device 320 (e.g., Rogowski coil, etc.) that may be communicatively coupled to an output device 324 (e.g., an oscilloscope, a computing device, a sensor, etc.), and a B-field measurement device 322 (e.g., Hall-effect sensor, etc.) that may also be communicatively coupled to an output device 326 (e.g., an oscilloscope, a computing device, a sensor, etc.). The PCB 304 may be configured to include one or more of the following: a charge/discharge controller 306, an energy storage device (e.g., a capacitor) 308, a voltage step up device (e.g., a boost converter) 310, a B-field generator device 312 (e.g., an inductive coil, as discussed above). An optional safety shutoff device 314 may also be incorporated onto the PCB 304. Moreover, a computing interface 316 may be coupled to the controller 316 and may be configured to control operation of the system 300, as discussed herein. Further, a power supply device (e.g., a 20V battery) 318 may be coupled to the voltage step up device 310 (and/or to any other component in the system 300). The enclosure 302 may be configured to protect components of the system 300 as well as users of the system 300, as the system 300 may be configured to operate a high voltage (e.g., >200 V) and a high current (e.g., >1.5 kA). Moreover, one or more switches (not shown in FIG. 3) may be included, e.g., for safety purposes (such as to prevent users from accidentally touching an energized capacitor or coil).

In some implementations, the capacitor 308 voltage V_{C}(t) may be measured using the output device 324 (e.g., oscilloscope). The current measurement device 320 may be configured to include a current waveform transducer to measure a high-speed current pulse I_{L}(t). For example, the current measurement device 320 may be wrapped around one end of the coil wire extending from the coil 312. The B-field measurement device 322 may be positioned at various distances from a center of a loop of the coil wire of the coil 312 to measure the magnetic field (B-field) strength along Z-axis. The sensor may be rotated by a predetermined angle (e.g., 90 degrees) to measure the B-field along X-axis at the center of the coil 312. The electric field may be measured in the X direction at several distances from the bottom of the coil 312. For example, the measurement may use a “pickup” coil that may include a wire (e.g., a straight wire, etc.) segment of a predetermined length Ls (e.g., 1.2 cm) that may be oriented along the direction of the electric field, and additional perpendicular wire segments that may extend beyond the region of induced electric field (and perpendicular to the induced electric field). The pickup coil may be connected to a high input impedance oscilloscope to determine the time-dependent coil voltage V(t), from which the electric field E(t)=V(t)/Ls may be determined.

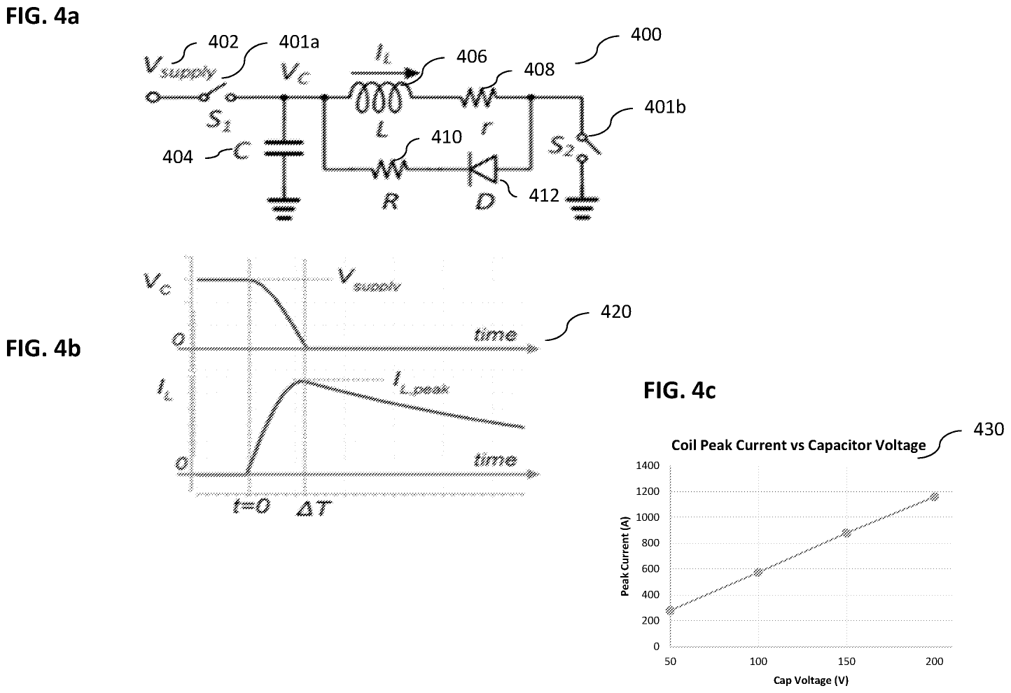

FIG. 4a illustrates an exemplary switching inductor-capacitor circuit 400 that may be used by the system 300 (shown in FIG. 3), according to some implementations of the current subject matter. As shown in FIG. 4a, the circuit 400 may include a power supply (V_{supply}) 402 (similar to power supply 318 shown in FIG. 3), a capacitor 404 (similar to the device 308 shown in FIG. 3), an inductor coil 406 (similar to device 312 shown in FIG. 3), one or more resistors (R, r) 408, 410, one or more switches 401 (a, b), and a diode 412.

In some implementations, the circuit 400 may be configured to generate high voltage (>200 V) and current (>1.5 kA), and may, for example, be enclosed in an enclosure (e.g., enclosure 302 shown in FIG. 3) with a lid fitted with momentary push-button switches for safety purposes. The switches formed a relay circuit such that as soon as the lid was opened, the voltage supply was disconnected and the capacitor immediately discharged. The capacitor 404 voltage V_{C}(t) may be measured using an external device (e.g., oscilloscope 326 shown in FIG. 3). A current measurement device (e.g., Rogowski coil 320 shown in FIG. 3) with a current waveform transducer may measure a high-speed current pulse I_{L}(t). For example, the current measurement device may be wrapped around one leg of the figure-8 (rTMS) coil. A B-field measurement device (e.g., Hall effect magnetic sensor 322 as shown in FIG. 3) may be positioned at various distances from the center of a coil loop to measure the magnetic field (B-field) strength along Z-axis. The same sensor may be rotated by 90 degrees to measure the B-field along X-axis at the center of the coil.

The capacitor 404 may be fully charged to the supply voltage (V_{supply}) 402 and then discharged through the inductor coil 406 thereby producing a peak current when the capacitor voltage drops to zero. This is shown by the diagram 420 in FIG. 4b. The energy stored in the electric field of the capacitor 404 (½ CV²) may be substantially the same as the energy stored in a magnetic field of the inductor 406 (½ LI²) at peak current. The peak inductor coil current may be determined using equations (4)-(5):

½CV²=½LI² (4)

Iₚₑₐₖ=V_{supply}/√(L/C) (5)

Using equation (5), the inductor current may be configured to increase with a supply voltage and capacitance, however, may decrease with the coil inductance. In some exemplary implementations, limitations on maximum supply voltage (V_{supply}) and capacitor size (C) may be imposed to ensure portability, safety and/or low power operation of the system. The rise time of the current pulse (ΔT) may be configured to be one quarter wave of the period of the LC resonant circuit formed between the coil and the storage capacitor, and may be determined using the following product (π/2)*√(LC). Thus, the current subject matter's inductor coil may be configured to generate small enough inductance to produce maximum peak current and/or short pulse width, while at the same time providing high magnetic field (which may increase with the number of turns in the coil). By way of a non-limiting, experimental example, the maximum operating voltage of the portable rTMS system shown in FIG. 4a may be 300V (or any other desired value). The capacitor may have capacitance of C=380 μF (or any other desired value). For a coil inductance of L=7.5 μH, the peak current may be Ipk=2.15 kA and the pulse width ΔT=84 μs. FIG. 4c illustrates an exemplary plot 430 showing exemplary experimental values determined using the above experimental system.

Referring back to FIG. 4a, the circuit 400 may be configured to operate as follows. First, at time t<0, switch S1 401a may be closed and switch S2 401b may be open. The capacitor (C) 404 may be charged to V_{supply} 402, thereby storing energy in its electric (E) field. Then, at time t>0, switch S1 401a may be open while switch S2 401b may be closed. The charged capacitor 404 may be configured to discharge through the inductor (L) 406. The capacitor voltage V_{C} may be configured to fall as the inductor current I_{L} rises. When capacitor C 404 is completely discharged (V_{C}=0V), all stored energy may be converted to magnetic field around the coil windings (i.e., as I_{L} peaks). A back electromotive force (EMF) may be induced in the coil 406 (V_{C}=L (·dI_{L})/dt), while keeping the current flowing (e.g., in the same direction) and re-charging the capacitor 404 (e.g., in the opposite polarity). This “oscillatory” behavior may repeat until energy is dissipated on the coil resistance (r) 408 as heat. As stated above, FIG. 4b illustrates exemplary transient voltage V_{C} and current I_{L} waveforms with zero or non-negligible resistive loss.

The voltage and current waveforms may be determined using equations (6) and (7):

$${V}_{C} ( t ) \approx {V}_{supply} \cdot {e}^{- \frac{r}{2 L} t} cos ( {ω}_{o} t ) \qquad ( 6 )$$

$${I}_{L} ( t ) \approx \frac{{V}_{supply}}{{ω}_{o} L} \cdot {e}^{- \frac{r}{2 L} t} sin ( {ω}_{o} t ) \qquad ( 7 )$$

where ωₒ is the resonance frequency (ωₒ1/√LC), and a high “quality factor” assumption may be made, i.e., ((ωₒL)/r)>>1. The pulse shape (i.e., sharpness) may be determined by ωₒ, while the rate of decay may be determined based on the value of resistor r 408. According to equation (7), the peak inductor current may be determined by:

$${I}_{L , peak} \approx \frac{{V}_{supply}}{{ω}_{o} L} = \frac{{V}_{supply}}{\sqrt{L / C}} \qquad ( 8 )$$

The peak current, which, in turn, gives rise to the peak magnetic field according to equation (1), may be maximized by performing at least one of the following: maximizing C, maximizing V_{supply}, and/or minimizing L. Further, the use of a small inductor may be balanced with an acceptable quality factor to ensure presence of inductor and/or magnetic field. Thus, for example, to keep the current pulse sharp, C=380 μF may be used, and assuming L=3 μH, ΔT≈π/((2ωₒ)=53 μs (where ΔT is defined in FIG. 4b). Using equation (8), if V_{supply}=150 V, then I_{L,peak}˜1.69 kA.

Equations (6)-(8) imply that successful bi-phasic magnetic pulse generation demands low resistive loss (small r). Otherwise, the magnitude of the negative current pulse may decay to a fraction of the positive one (e.g., the dotted lines in FIG. 4b). To keep r small, the coil and connecting cables may be include sufficiently thick copper wires, which may make making the rTMS prototype heavier and bulkier. Thus, mono-phasic pulse generation may be used to achieve higher portability (as shown in FIG. 4a). In some implementations, as shown in FIG. 4a, the mono-phasic rTMS prototype may include the flyback diode (D) 412 that may clamp V_{C} to no more than 1 diode drop below 0V, thereby protecting the capacitor 404. The diode 412 may be configured to direct current I_{L} to a resistor R 410, which may dissipate the stored energy. At that point, charging may happen again (i.e., S₁/S₂ 401 (a, b) being closed/open, respectively) to generate the next magnetic pulse (S₁/S₂ being open/closed, respectively) for repetitive TMS application.

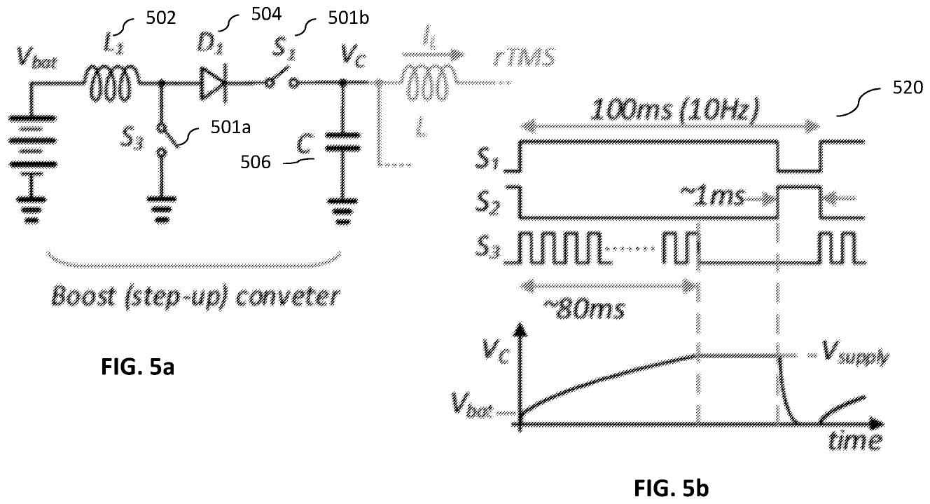

FIG. 5a illustrates an exemplary voltage step-device, e.g., a boost DC-DC converter 500, according to some implementations of the current subject matter. The device 500 may be similar to the device 310 shown in FIG. 3. The device 500 may include an inductor L₁ 502, a diode Di 504, a capacitor C 506, and one or more switches S₁ 501a and S₃ 501b. The device 500 may be configured to enable operation of the current subject matter's rTMS device (e.g., device 100 shown in FIG. 1, device 300 shown in FIG. 3). The device 500 may be configured to step up the low battery voltage Vbat to produce a much higher V_{supply} (as discussed above). During operation of the device 500, the switch S₃ 501b may be configured to be toggled between on and off positions rapidly. When the switch S₃ is closed, the compact inductor (L₁) 502 may be connected between Vbat and ground, thereby developing a current (e.g., stored magnetic energy) that may grow with time (I_{L1}=(Vbat Δt/L₁), where Δt is the time during which the switch S₃ 501b is closed. When switch S₃ 501b is open, the inductor voltage may be reversed to maintain the current towards the diode Di 504. The energy stored on the inductor 502 may be transferred to the capacitor C 506, thereby charging it (inductor L₁ 502 may be assumed to be fully discharged per cycle). When switch S₃ 501b is closed again for inductor 502 charging, the diode 504 may block the capacitor 506 from discharging. This process may repeat until the target V_{supply} is reached.

For example, assuming L₁=10 pH and C=380 g, it may take approximately 8000 cycles (80 ms) of switch S₃ switching at 100 kHz to boost the voltage from 20V (e.g., Vbat) to 170 V (V_{supply}). This is within the rTMS repetition rate of 10 Hz (or 100 ms period). FIG. 5b illustrates an exemplary timing diagram 520 illustrating the above example (with switch S₁ (shown in FIG. 5a), switch S₂ (shown in FIG. 4a), and switch S₃ (shown in FIG. 5a) being opened/closed at predetermined times). In some implementations, the boost converter and the rTMS circuit may be placed on separate PCBs (e.g., for more efficient debug, evaluation, etc.).

### Example Experiments

In one exemplary experiment, a compact, battery-powered high current (1.5 kA) rTMS device (in accordance with some of the exemplary implementations discussed above) that can repetitively generate 3,000 magnetic pulses of 0.1 Tesla over 10 minutes with a pulse rise time of less than 70 μs was used. This pulse speed may be sufficient to elicit physiological responses in patient's brain cortical neurons, assuming adequate field strength. During the experiment, the current subject matter device generated a magnetic field of 0.1 Tesla which is on the same order of magnitude as typical therapeutic levels (0.3 to 2 T). It should be noted that lower field intensities in the range of 0.01-0.1 T may affect neuron resting membrane potential and action-potential threshold. As such, low-intensity magnetic and electric fields have alleviated depression in humans. Nonetheless, for safety and practical testing considerations field strength was kept to 0.1 Tesla, but the driving circuitry of the current subject matter device supported up to 0.6 Tesla.

An objective of the experiment was to show that the current subject matter rTMS device (having a fully-charged L₁-ion battery) may generate magnetic pulse train with enough (1) magnitude, (2) frequency and/or (3) duration to achieve the desired neuro-therapeutic effects in the patient. The device may be portable (or even wearable), reasonably lightweight and/or compact. In one experiment, a low power, voltage scalable test platform was used to generate magnetic pulses with one or more of the following characteristics: with 0.1 Tesla peak magnetic flux density (B) at 2 cm depth, at the maximum rate of 11 Hz, for a total of 2000-3000 pulses per treatment session. The pulses may be short (e.g., less than 200 μs) with rapid rise time (e.g., for maximum dB/dt) in order to produce rapidly changing magnetic fields and corresponding electric fields necessary to trigger neurophysiological effects. The pulses may be monophasic.

To characterize the rTMS device, the capacitor voltage, the inductor current, and the B-field were measured at the 10 Hz pulse firing rate. The battery at full charge was verified to deliver the energy needed to repeatedly charge the capacitor from 0V to the target V_{supply} for at least 2000 cycles at 10 Hz.

Since the rTMS device functioning involves high voltage (>150 V) and current (>1.5 kA), it may be fully enclosed in a Plexiglass box with a lid fitted with momentary push-button switches for safety purposes. The switches form a relay circuit such that as soon as the lid is open, the voltage supply will be disconnected from the device, and the rTMS capacitor immediately discharged. This is to prevent users from accidentally touching an energized capacitor or coil, as discussed above, in connection FIGS. 3-5b.

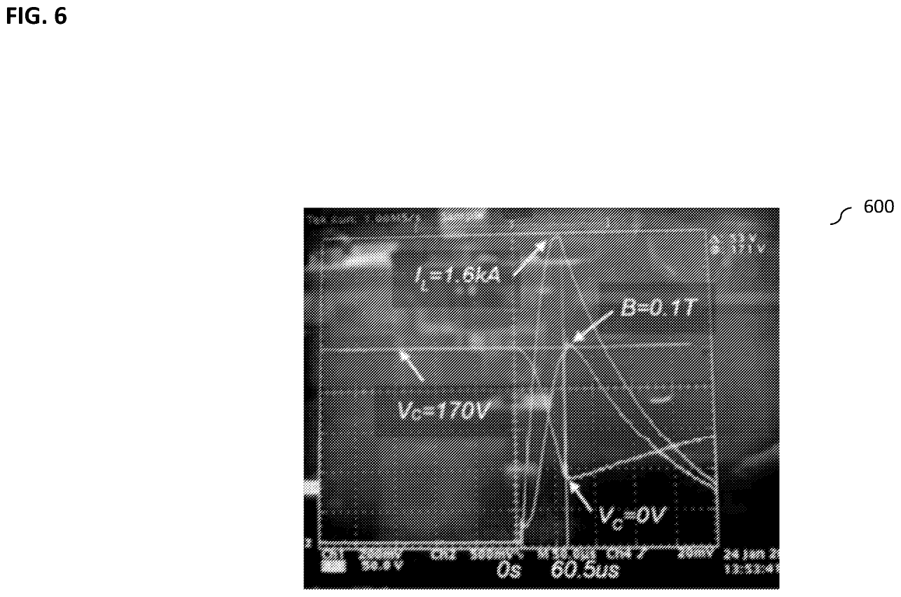

FIG. 6 illustrates exemplary experimental measured transient waveforms for V_{C}(t), I_{L}(t) and the B-field 600, generated in accordance with implementations of the current subject matter system. For example, for V_{supply} of 170 V, the peak current reaches 1608 A at 60.5 μs (after switches S₁ 401a and S₂ 401b (as shown in FIG. 4a) may be flipped open and closed, respectively). This may correspond to the capacitor 404 voltage dropping from its peak (170 V) to approximately 0V. At that instance, the peak magnetic field of 100 mT may be measured by the Hall sensor (e.g., sensor 322 shown in FIG. 3) at 2 cm distance. At time >60.5 μs, the inductor (e.g., inductor 406 shown in FIG. 4a) current may be routed to the resistor R (e.g., resistor R 410 as shown in FIG. 4a) through the flyback diode D (e.g., diode 412). The stored magnetic energy may be dissipated as heat on the resistance. At the same time, the capacitor may be re-charged by the external power supply, thereby being ready for the next pulse firing. The current subject matter system was tested for 5 minutes at the repetition rate of 10 Hz (3000 pulses) and sustained and stable operation was confirmed.

In another experimental implementations, a battery-powered compact rTMS device weighing approximately 12.6 gm (0.4 oz), having 76×38 mm figure-8 inductor coil (in accordance with the implementations discussed above) was tested and generated an E-field of 65 V/m at 1.5 cm. This E-field intensity may be sufficient to engage brain cortical regions at 1.5 cm distance from the scalp. In particular, two experimental figure-8 inductor coils, having loop inner/outer diameters of 2.5 cm/3.1 cm and 3.6 cm/3.8 cm, total lengths of 6.2 cm and 7.6 cm, and 9 and 6 turns, respectively, were tested. The 76×38 mm coil (i.e., 7.6 cm total length and 3.8 cm outer diameter) weighed 12.6 gm (0.4 oz), generated an E-field of 65 V/m (which is in contrast to 1.8-3.9 Kg (4-8.6 lbs) of existing rTMS head coils), and at 1.5 cm distance, induced 65% of the E-field intensity of conventional systems operating at 60% power. The inductance of the 9-turn coil (62×31 coil) was 7.83 pH and the inductance of the 6 turn coil (76×38) was 3.89 pH. The experimental current peak values for the smaller coil were between 880 A and 1160 A. The experimental current peak values for the larger coil were between 1680 A and 2480 A. The current subject matter test system was operated at low power but higher electric fields were attained with higher supply voltages.

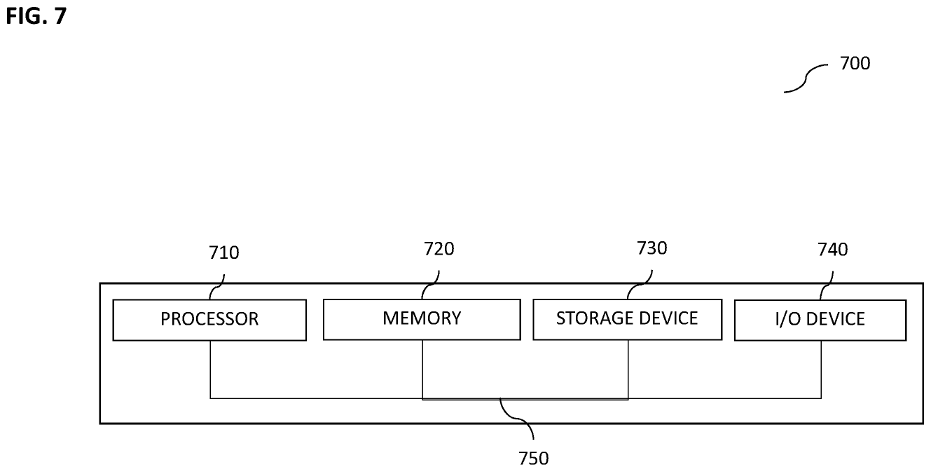

In some implementations, the current subject matter can be configured to be implemented and/or operating in connection with a computing system 700, as shown in FIG. 7. The system 700 can include a processor 710, a memory 720, a storage device 730, and an input/output device 740. Each of the components 710, 720, 730 and 740 can be interconnected using a system bus 750. The processor 710 can be configured to process instructions for execution within the system 700. In some implementations, the processor 710 can be a single-threaded processor. In alternate implementations, the processor 710 can be a multi-threaded processor. The processor 710 can be further configured to process instructions stored in the memory 720 or on the storage device 730, including receiving or sending information through the input/output device 740. The memory 720 can store information within the system 700. In some implementations, the memory 720 can be a computer-readable medium. In alternate implementations, the memory 720 can be a volatile memory unit. In yet some implementations, the memory 720 can be a non-volatile memory unit. The storage device 730 can be capable of providing mass storage for the system 700. In some implementations, the storage device 730 can be a computer-readable medium. In alternate implementations, the storage device 730 can be a floppy disk device, a hard disk device, an optical disk device, a tape device, non-volatile solid state memory, or any other type of storage device. The input/output device 740 can be configured to provide input/output operations for the system 700. In some implementations, the input/output device 740 can include a keyboard and/or pointing device. In alternate implementations, the input/output device 740 can include a display unit for displaying graphical user interfaces.

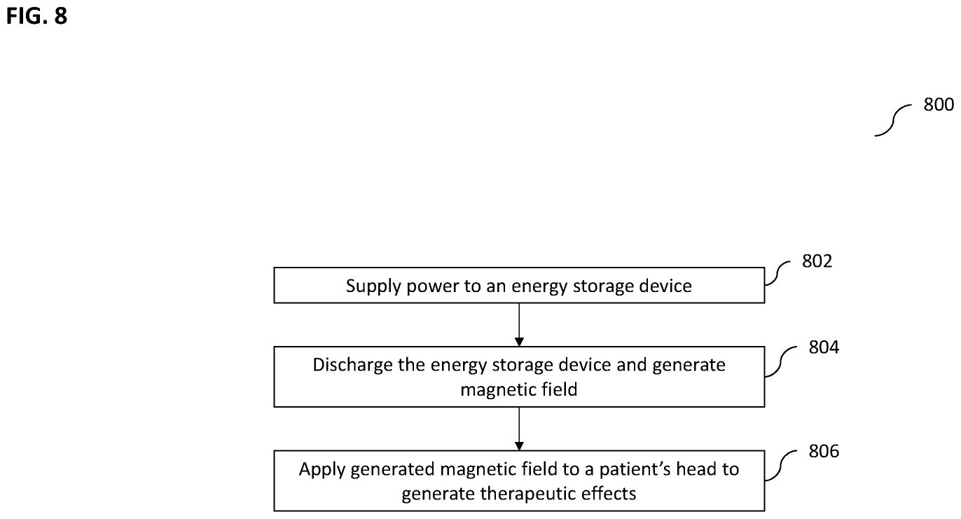

FIG. 8 illustrates an exemplary process 800 for performing repetitive transcranial magnetic stimulation of a brain of a user, according to some implementations of the current subject matter. The process 800 may be performed using the rTMS system 100 shown in FIG. 1 and/or system 300 shown in FIG. 3 (including the components of these systems as shown in FIGS. 2, 4a-5b). By way of a non-limiting example, the process 800 may be configured to be performed by a battery-powered compact rTMS prototype capable of rapidly and repeatedly generating 100 mT magnetic fields at 1.5-2.5 cm depth, and driving circuitry scalable to higher power levels. The process 800 may be configured to be part of various therapeutic procedures to treat various neuropsychiatric and addictive disorders in various settings (e.g., home, clinic, hospital, medical facility, field hospital, doctor's office, orbital space stations, etc.).

At 802, a power from voltage source may be supplied to an energy storage device (e.g., capacitor 308 shown in FIG. 3) for charging/energy storage. The voltage may be supplied to the capacitor 308 via a voltage step up device (e.g., boost converter 310 shown in FIG. 3). The generated voltage may be on the order of greater than 200 V (e.g., 200V-400V), for example.

At 804, the capacitor may be configured to discharge, thereby causing the B-field generator (e.g., the inductive coil 312 shown in FIG. 3) to generate a magnetic field. At 806, the magnetic field may be applied to the patient's head (e.g., via an adaptor, a helmet, etc.) for a predetermined period of time to provide therapeutic effects.

In some implementations, the current subject matter relates to a portable therapeutic device (e.g., devices discussed above and shown in FIGS. 1-6). The device may include an energy storage device (e.g., capacitor 308 shown in FIG. 3, capacitor 404 shown in FIG. 4a) coupled to a power supply (e.g., battery 318, power supply 402). The energy storage device may be configured to store energy received from the power supply. The energy storage device may be further configured to operate during a predetermined number of charge-discharge cycles. During a charge portion of each charge-discharge cycle, the energy storage device may be configured to receive and store energy from the power supply. During a discharge portion of each charge-discharge cycle, the energy storage device may be configured to discharge stored energy. The portable therapeutic device may be configured to accommodate currents of at least 800 Amperes (e.g., 800-2500 A) and a voltage supply of at least 200 Volts (e.g., 200-400 V).

The portable therapeutic device may further include a magnetic field generation device (e.g., inductive coil 312; inductive coil 406) that may be coupled to the energy storage device and configured to repeatedly generate one or more magnetic field pulses in a plurality of magnetic field pulses during a predetermined period of time (e.g., 13-15 minutes). Each magnetic field pulse may have a predetermined magnetic field strength. The pulses may be generated during the discharge portion of each charge-discharge cycle of the energy storage device. Further, the pulses may include single phasic and/or biphasic magnetic pulses occurring as pulse trains over a predetermined frequency (e.g., 10 Hz, etc.) over a predetermined period of time (e.g., 10 seconds, etc.), each pulse train including a predetermined number of pulses (e.g., 100 pulses), where trains may include bursts that may be separated by a predetermined period of time, as discussed above. The therapeutic device may be configured to generate one or more (e.g., 20) pulse trains separated by a predetermined inter-train interval (e.g., 30 seconds, etc.). For example, the therapeutic device may be able to generate up to 4000 pulses or more during any therapeutic/treatment time period (e.g., 13-15 minutes).

The generated magnetic field pulses may be configured to cause generation of an electric field having a predetermined strength, thereby generating a desired therapeutic effect in a subject.

In some implementations, the current subject matter may be configured to include one or more of the following optional features. As stated above, the energy storage device may include a capacitor. The magnetic field generation device may include an inductive coil having a conductive wire, the conductive wire is configured to be wound. The inductive coil may be configured to have a predetermined shape. The predetermined shape may include at least one of the following: a circular shape, a figure-8 shape, an oval shape, an elliptical shape, a butterfly shape, a double butterfly shape, a triple butterfly shape, an H-coil shape, a regular shape, an irregular shape, and any combination thereof.

In some implementations, the inductive coil may include at least one of the following parameters: a predetermined length, a predetermined number of winding turns of the conductive wire, a predetermined radius of one or more winding turns of the conductive wire, a thickness of the conductive wire, and any combination thereof. The predetermined magnetic field strength may be determined using at least one of the inductive coil parameters. The predetermined length may be in a range of approximately 50 mm to 150 mm.

In some implementations, the predetermined strength of the generated electric field may be a range of approximately 50 V/m to 120 V/m. In particular, the predetermined strength of the generated electric field may be approximately 65 V/m.

In some implementations, the power supply may be rechargeable.

In some implementations, the magnetic field generation device may be configured to generated one or more magnetic field pulses as a result of a predetermined current received from the energy storage device. The predetermined current may be in a range of approximately 800 A to 2500 A.

In some implementations, the magnetic field pulses may be generated at a predetermined frequency, where the predetermined frequency may be determined based on the desired therapeutic effect.

In some implementations, the therapeutic device may include a voltage step-up device coupled to the power supply and the energy storage device and configured to increase voltage being supplied by the power supply to the energy storage device. The voltage supplied to the energy storage device may be greater than approximately 200 V.

In some implementations, the therapeutic device may include a printed circuit board for positioning at least one of the power supply, the energy storage device, the magnetic field generation device, and any combination thereof.

In some implementations, the magnetic field pulses may be configured to be applied to the subject from a predetermined distance. The predetermined distance may be in a range of 1.5 cm to 2.5 cm. Further, the therapeutic effect may include a repetitive transcranial magnetic stimulation. The predetermined magnetic field strength may be greater than 100 mT.

In some implementations, the current subject matter may relate to a method for providing repetitive transcranial magnetic stimulation to a subject. The method may include providing the portable therapeutic device discussed above, repeatedly generating, using the magnetic field generation device, one or more magnetic field pulses, and providing the repeatedly generated magnetic field pulses to a subject, and causing generation of an electric field having a predetermined strength, thereby generating a desired therapeutic effect in the subject.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively, or additionally, store such machine instructions in a transient manner, such as for example, as would a processor cache or other random access memory associated with one or more physical processor cores.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A portable therapeutic device, comprising:
an energy storage device coupled to a power supply and configured to store energy received from the power supply, the energy storage device being configured to operate during a predetermined number of charge-discharge cycles, wherein during a charge portion of each charge-discharge cycle, the energy storage device is configured to receive and store energy from the power supply, and during a discharge portion of each charge-discharge cycle, the energy storage device is configured to discharge stored energy;
a magnetic field generation device coupled to the energy storage device and configured to repeatedly generate one or more magnetic field pulses in a plurality of magnetic field pulses during a predetermined period of time, each magnetic field pulse having a predetermined magnetic field strength, during the discharge portion of each charge-discharge cycle of the energy storage device;
the generated magnetic field pulses being configured to cause generation of an electric field having a predetermined strength, thereby generating a desired therapeutic effect in a subject.

2. The portable therapeutic device according to claim 1, wherein the energy storage device includes a capacitor.

3. The portable therapeutic device according to claim 1, wherein the magnetic field generation device includes an inductive coil having a conductive wire, the conductive wire is configured to be wound.

4. The portable therapeutic device according to claim 3, wherein the inductive coil is configured to include a predetermined shape.

5. The portable therapeutic device according to claim 4, wherein the predetermined shape includes at least one of the following: a circular shape, a figure-8 shape, an oval shape, an elliptical shape, a butterfly shape, a double butterfly shape, a triple butterfly shape, an H-coil shape, a regular shape, an irregular shape, and any combination thereof.

6. The portable therapeutic device according to claim 3, wherein the inductive coil includes at least one of the following parameters: a predetermined length, a predetermined number of winding turns of the conductive wire, a predetermined radius of one or more winding turns of the conductive wire, a thickness of the conductive wire, and any combination thereof.

7. The portable therapeutic device according to claim 6, wherein the predetermined magnetic field strength is determined using at least one of the inductive coil parameters.

8. The portable therapeutic device according to claim 6, wherein the predetermined length is in a range of approximately 50 mm to 150 mm.

9. The portable therapeutic device according to claim 1, wherein the predetermined strength of the generated electric field is in a range of approximately 50 V/m to 120 V/m.

10. The portable therapeutic device according to claim 1, wherein the predetermined strength of the generated electric field is approximately 65 V/m.

11. The portable therapeutic device according to claim 1, wherein the power supply is rechargeable.

12. The portable therapeutic device according to claim 1, wherein the magnetic field generation device is configured to generate one or more magnetic field pulses as a result of a predetermined current received from the energy storage device.

13. The portable therapeutic device according to claim 12, wherein the predetermined current being in a range of approximately 800 A to 2500 A.

14. The portable therapeutic device according to any of the preceding claims, wherein the one or more magnetic field pulses are generated at a predetermined frequency, the predetermined frequency being determined based on the desired therapeutic effect.

15. The portable therapeutic device according to claim 1, further comprising a voltage step-up device coupled to the power supply and the energy storage device and configured to increase voltage being supplied by the power supply to the energy storage device.

16. The portable therapeutic device according to claim 15, wherein the voltage supplied to the energy storage device is greater than approximately 200 V.

17. The portable therapeutic device according to claim 1, further comprising a printed circuit board for positioning at least one of the power supply, the energy storage device, the magnetic field generation device, and any combination thereof.

18. The portable therapeutic device according to claim 1, wherein the magnetic field pulses are being configured to be applied to the subject from a predetermined distance.

19. The portable therapeutic device according to claim 18, wherein the predetermined distance is in a range of 1.5 cm to 2.5 cm.

20. The portable therapeutic device according to claim 1, wherein the therapeutic effect include a repetitive transcranial magnetic stimulation.

21. The portable therapeutic device according to claim 1, wherein the predetermined magnetic field strength is greater than 100 mT.

22. A method comprising:
providing a portable therapeutic device having an energy storage device coupled to a power supply and configured to store energy received from the power supply, the energy storage device being configured to operate during a predetermined number of charge-discharge cycles, wherein during a charge portion of each charge-discharge cycle, the energy storage device is configured to receive and store energy from the power supply, and during a discharge portion of each charge-discharge cycle, the energy storage device is configured to discharge stored energy;
a magnetic field generation device coupled to the energy storage device and configured to repeatedly generate one or more magnetic field pulses in a plurality of magnetic field pulses during a predetermined period of time, each magnetic field pulse having a predetermined magnetic field strength, during the discharge portion of each charge-discharge cycle of the energy storage device;
repeatedly generating, using the magnetic field generation device, the one or more magnetic field pulses; and
providing the repeatedly generated magnetic field pulses to a subject, and causing generation of an electric field having a predetermined strength, thereby generating a desired therapeutic effect in the subject.
